(19) United States
(12) Patent Application Publication
(10) Pub. No.: US 2011/0137425 A1
(43) Pub. Date: Jun. 9, 2011

(54) TOTAL KNEE PROSTHESIS AND RANGE OF ELEMENTS FOR PRODUCING SAID PROSTHESIS

(76) Inventors: Michel Allard, Pessac (FR); Vincent Bousquet, Latresne (FR); Philippe Colombet, Latresne (FR); Christophe Delavigne De Sainte Suzanne, Bruges (FR)

(21) Appl. No.: 12/524,806

(22) PCT Filed: Oct. 4, 2005
(86) PCT No.: PCT/FR05/02432

§ 371 Date: Jan. 4, 2010

(30) Foreign Application Priority Data
Oct. 7, 2004 (FR) 0410573

(51) Int. Cl. A61F 2/38

(52) U.S. Cl. 623/20.21

(57) ABSTRACT

The invention concerns a prosthesis (1) comprising a femoral implant (2) and a tibial implant (3) equipped with anchoring rods (21), each anchoring rod (21) not being integral with the corresponding implant and capable of being connected thereto, the prosthesis (1) including therefor means (16, 20; 36, 34) for assembling each anchoring rod (21) to the corresponding implant (2, 3). The invention is characterized in that the means (16, 20; 36, 34) for assembling each medullar anchoring rod (21) to the corresponding implant (2, 3) are configured to allow a plurality of possible angular positions of that same anchoring rod (21) relative to the implant (2, 3) in translation along an axis inscribed in the frontal plane, that is lateral, or in the sagittal plane, that is antero-posterior, of the implant.

The present invention relates to a total knee prosthesis and a series of components enabling composition of this prosthesis.

In certain cases, arthroplasties of the knee are particularly difficult to achieve, the ligaments of the joint being faulty, even inoperative, and/or the quality of bone epiphyses being insufficient to enable a stable support of the femoral implant and of the tibial implant.

Among the existing knee prostheses, certain ones, called “joint”, comprise a bicondylar femoral implant, a femoral implant mechanically connected to the tibial implant and pivotably guided relative to it about a determined transverse axis, and an intermediate fixed plateau made of polyethylene.

These prostheses have the disadvantages of not being modular and not enabling restoration of a mechanically stable joint with a favorable distribution of loads. Instead, these prostheses induce significant stresses at the joint surfaces and bone anchors.

Another type of “joint” prosthesis comprises a tibial implant equipped with a longitudinal stud on which is fitted a tubular component connected to the femoral implant, this fitting enabling stabilization of the femur and the tibia in the sagittal plane and thus making up for the deficiencies of the ligaments. The implants of these prostheses are equipped with fixed anchoring rods, which have a disadvantage of generating significant stresses localized on the longitudinal stud and on the bone anchors. It results in risks of premature wear of the prosthesis and risks of loosening of the implants.

Yet another type of “joint” prostheses, more developed, comprises a polyethylene plateau designed to distribute stresses on the two plateau/femoral implant and plateau/tibial implant interfaces. However, the significant possibility of rotation of the tibial plateau induces an instability of the prosthesis when the lower member is extended.

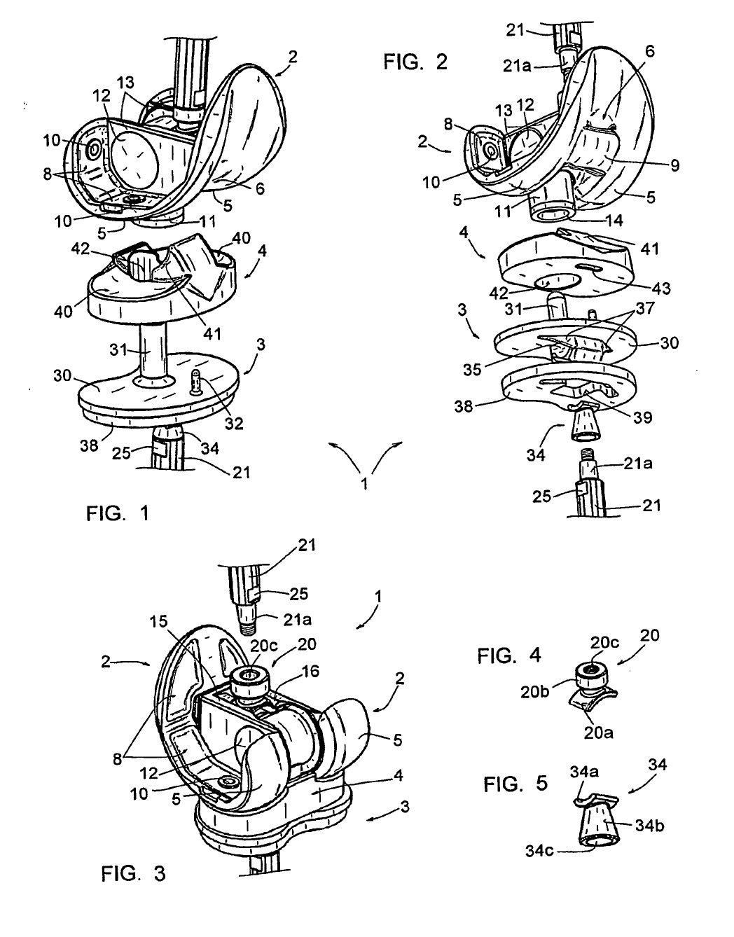

Document U.S. Pat. No. 5,879,391 describes a knee prosthesis with femoral and tibial anchoring rods adjustable in position. The femoral anchoring rod is adjustable in translation in the sagittal plane (FIGS. 1 to 6) and the tibial anchoring rod is adjustable in translation in the frontal plane (FIGS. 7 to 10).

Document U.S. Pat. No. 5,236,359 describes a bone implant comprising a transverse slide 15 in a dovetail, a base 2 slidably engaged in this slide and an anchoring rod connected to the base. The slide can be longitudinal.

The adjustments in position contemplated by these documents do not enable effective remedy of the aforesaid risks of premature wear of the prosthesis and loosening of the implants.

The present invention aims to solve this problem is, generally, for remedy of the drawbacks of existing prostheses.

Its principal objective is to enable the reconstruction of a knee joint by means of a prosthesis fully adaptable to the specific anatomy of the joint to be treated and thus having a reduced risk of wear and loosening.

Another objective of the invention is to provide such a prosthesis, adapted to a difficult arthroplasty, that is to say when the joint ligaments are deficient, even inoperable, and/or when the joint has epiphyseal deformities or bone gaps caused by trauma or previous surgeries.

An additional objective of the invention is to provide such a prosthesis, having a high stability when the lower member is extended.

An additional objective of the invention is to provide a series of modular components enabling composition of a prosthesis fully adapted to the specific anatomy of the joint to be treated.

The prosthesis concerned comprises, in a manner known per se, a femoral implant and a tibial implant equipped with medullar anchoring rods and an intermediate plateau made of material promoting sliding, in particular, made of high density polyethylene, each medullar anchoring rod of an implant is not integral with this implant and is connectable to it, the prosthesis comprising to this end means for the coupling of each medullar anchoring rod to the corresponding implant and tightening means to immobilize each medullar anchoring rod relative to the corresponding implant.

According to the invention, the means that the prosthesis comprises for the coupling of each medullar anchoring rod to the corresponding implant are designed to enable a plurality of possible angular positions of the medullar anchoring rod relative to the implant and a plurality of possible positions of this same medullar anchoring rod relative to the implant in translation along an axis contained in the frontal plane, that is to say lateral, or in the sagittal plane, that is to say anteroposterior, of the implant.

Due to this double adjustability of the position of a medullar anchoring rod relative to the corresponding implant, it is possible to accurately position the anchoring rods relative to the femoral and tibial implants according to the specific anatomy of the patient and thus obtain an optimal functioning of the prosthesis regardless of this anatomy. The prosthesis consequently has greatly reduced risks of wear and loosening.

Preferably, the means for the coupling of the femoral anchoring rod to the femoral implant are designed to enable angular adjustment of the anchoring rod in the frontal plane and the adjustment in translation of this rod in the sagittal plane.

Preferably, the means for the coupling of the tibial anchoring rod to the tibial implant are designed to enable angular adjustment of the anchoring rod in the sagittal plane and the adjustment in translation of this rod in the frontal plane.

According to a preferred from of implementation of the invention,
at least one anchoring rod is designed to be connected to the corresponding implant by an intermediate coupling part;
this intermediate coupling part has means for the coupling of the rod to it and a curved end wall;
the implant has a cavity for reception and retention of the aforementioned curved end wall, having the same profile as the aforementioned curved end wall and the bottom of which is delimited by a curved reception wall against which comes to bear the aforementioned curved end wall at the time of the coupling; this curved reception wall has an arc length greater than that of the aforementioned curved end wall, so that this curved end wall, and thus the aforementioned anchoring rod connected to it, can be positioned according to several angular positions relative to the implant; the cavity is shaped along an axis contained in the frontal plane or in the sagittal plane of the implant so that it enables the sliding of the aforementioned curved end wall in it in order to carry out the adjustment in translation of the medullar anchoring rod relative to the implant.

Advantageously, the means for the coupling of a medullar anchoring rod to an intermediate coupling part comprise:
a threaded bore longitudinally traversing the intermediate anchoring part and opening into the concave face of the aforementioned curved end wall;
a threaded rod integral with the medullar anchoring rod, adapted to be screwed into the aforementioned threaded bore, this rod having a length slightly greater than that of the bore so as, in coupling position of the medullar anchoring rod to the intermediate coupling part, to bear against the aforementioned curved reception wall and thus tighten the intermediate coupling part against the walls of the implant delimiting the aforementioned cavity for reception and retention of the aforementioned curved end wall.

This bore, this threaded rod and these walls of the implant thus also forming the aforesaid tightening means enabling immobilization of a medullar anchoring rod relative to the corresponding implant.

In practice, consequently, the medullar anchoring rod is connected by screwing to the intermediate coupling part without complete tightening, then the position of the medullar anchoring rod is adjusted; the complete tightening of the medullar anchoring rod relative to the intermediate coupling part is then carried out to immobilize this rod and this part relative to the implant.

The prosthesis according to the invention can, in particular, be a prosthesis with limited movement of the femoral implant relative to the tibial implant in translation in the sagittal plane, such as a prosthesis called “posterior-stabilized”, that is to say comprising stops for limiting the aforementioned translation, or a prosthesis called “joint”, that is to say comprising means for mounting of the femoral implant relative to the tibial implant reducing the possibility of movement of the femoral implant relative to the tibial implant with a pivoting according to a determined transverse axis.

The aforesaid double adjustability enables the use of such implants with restricted movement of the femur, without exercise of significant strains on the joint areas from the femoral implant to the tibial implant or on the bone anchor areas.

The prosthesis according to the invention can be a prosthesis with two condyles; preferably, it is a prosthesis with three condyles, that is to say having, at the femoral implant and the intermediate plateau, curved support surfaces arranged at the middle, between the two lateral condyles.

The intermediate plateau can be fixed on the tibial implant, or may be movable relative thereto. Preferably, in this second case, that means are provided to limit the pivoting of the intermediate plateau relative to the tibial implant, in particular, a stud protruding from the plateau that forms the tibial implant and a groove in a circular arc centered on the pivot axis of the intermediate plate, arranged in this intermediate plate, this groove receiving the aforementioned stud and cooperating with it to limit the pivoting of the intermediate plateau relative to the tibial implant.

Advantageously, the femoral implant and/or tibial implant comprise means for mounting on them one or several shims or spacers enabling compensation for possible bone ablations or absences at the support surfaces of the implants against the femur and/or tibia.

The series of components of the invention comprises:
at least one femoral implant, at least one tibial implant and at least one intermediate plate, and preferably the femoral and tibial components of different sizes having connecting means for the medullar anchoring rods of identical dimensions and shapes of one component to the other;
at least two intermediate coupling parts;
at least two medullar anchoring rods, and preferably more than two medullar rods anchor, having different diameters, lengths, shapes and, these medullar anchoring rods being assembled to the aforementioned intermediate coupling parts

The modularity of these components enables implementation of a prosthesis fully adapted to the specific anatomy of the joint to be treated.

The aforementioned series can also comprise shims or spacers for compensation of bone ablations or absences, having different thicknesses, adapted to the reconstruction of adequate support surfaces for the femoral and tibial implants.

The invention will be well understood, and other features and advantages thereof will become apparent, with reference to the attached schematic drawing, representing, as non-limiting example, a preferred implementation of the prosthesis to which it relates.

FIG. 1 is a perspective view, before assembly;

FIG. 2 is a perspective view, before assembly, from another angle,

FIG. 3 is a perspective view, under assembly;

FIGS. 4 and 5 are perspective views of two intermediate coupling parts that comprise the prosthesis;

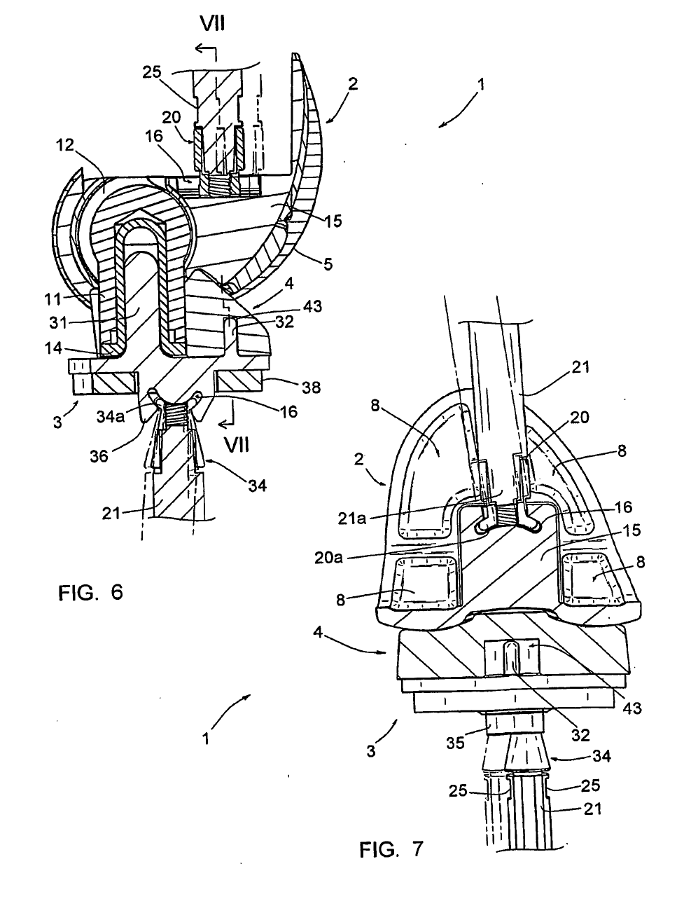

FIG. 6 is a median sectional view in the sagittal plane of the prosthesis after assembly, and

FIG. 7 is a sectional view along the line VII-VII in FIG. 6, in the frontal plane, of the prosthesis.

The figures show a total knee prosthesis 1, comprising, as is well known, a femoral implant 2, a tibial implant 3 and an intermediate plateau 4.

The femoral implant 2 has an enveloping “shield” shape, defining on its exterior face two lateral condyles 5 and a prosthetic trochlea 6 and, on its interior face, facets 8 for support against the end of the duly resected bone. In the example shown, the femoral implant 2 also has a central condyle 9, enabling increase of the surface for support of the femoral implant 2 against the intermediate plateau 4. It also comprises tapped blind holes 10, opening at distal and posterior facets 8, for mounting of one or several shims or spacers (not shown), which enable compensation for possible bone ablations or absences at the surfaces for support of the implant 2 against the femur.

The prosthesis 1 is of the type called “joint” in which the femoral implant 2 is mechanically connected to the tibial implant 3 and is guided by pivoting relative to the latter about a transverse axis determined in order to make up for the deficiencies of the ligaments of the joint of the patient. To this end, the femoral implant 2 comprises a rear socket 11 pivotally mounted relative to it by means of pivots 12 engaged in bearings, these bearings being arranged in two longitudinal bulkheads 13 that form the femoral implant 2 on its interior face. The socket 11 is axially drilled and receives a packing 14 made of a material promoting sliding, in particular made of high density polyethylene.

As FIGS. 3, 6 and 7 show it more particularly, the femoral implant 2 forms, in front of walls 13, a solid block 15 in which is fitted in a groove 16 opening at the proximal face and at the posterior face of the block 15. This groove 16 is shaped according to an axis contained in the sagittal plane of the implant 2 and has a circular arc shape in its lower part and a rectangular shape in its upper part, the upper part being narrower than the lower part.

The groove 16 is designed to receive an intermediate coupling part 20 of the medullar anchoring rod 21 of the femoral implant 2 to the femur.

The part 20 is more particularly visible in FIG. 4. It comprises a curved end wall 20a, made in a circular arc, having an arc length less than that of the wall delimiting the bottom of the groove 16, a proximal boss 20b, whose base has a width less than that of the upper part of the groove 16, and a bore 20c arranged along the axis of the boss 20b, piercing the part 20 from side to side and tapped at the base of the boss 20b and at the wall 20a.

The wall 20a and the boss 20b are designed to be respectively engaged in these upper and lower parts of the groove 16, with the possibility of sliding in the sagittal plane, as shown in dotted lines in FIG. 6, and with the possibility of pivoting in the frontal plane, as shown in dotted lines in FIG. 7.

As for the medullar anchoring rod 21, it has a distal portion 21a designed to be engaged in the bore 20c, threaded at its end and having a length slightly greater than that of the bore 20c. As can be understood in reference to FIGS. 6 and 7, the rod 21-part 20 assembly can slide and pivot in the groove 16 before complete tightening of the rod 21 relative to the part 20, and is locked in position after complete tightening of this rod 21 relative to the part 20, the distal portion 21a coming to bear in this position against the rounded boss delimiting the bottom of the groove 16 and consequently tightening the curved wall 20a against the edges of the block 15 delimiting the upper part of this groove. It is thus possible to adjust the position of the medullar rod 21 relative to the femoral implant 2 by anteroposterior sliding and by tilting in the frontal plane and then immobilizing this rod 21 in the selected position.

For its tightening, the rod 21 has two flat sections 25 enabling the engagement of a suitable tightening tool.

The tibial implant 3 forms a plateau 30 designed to recreate the upper plateau of the tibia. On its proximal face, this plateau 30 has a posterior stud 31 designed to be received, in an adjustable manner, in the packing 14 of the socket 11 and an anterior stud 32 designed to cooperate with a groove 43 of the intermediate plateau 4. On its distal face, the plateau 30 has a block 35 integral with it, in which is arranged a groove 36 and lateral walls 37 for reinforcement and setting relative to the bone. The groove 36 extends in a medial direction and is shaped, and has a shape similar to that of the groove 16.

As FIGS. 2 and 6 show it more particularly, the tibial implant 3 receives a medullar anchoring rod 21 identical to the medullar anchoring rod 21 of the femoral implant 2, by means of an intermediate coupling part 34 similar to the part 20. As it is shown more particularly in FIG. 5, this part 34 comprises a curved wall 34a, a conical boss 34b and a partially threaded bore 34c. This part 34 and the groove 36 enable an adjustment of the position of the rod 21 in inclination in the sagittal plane, as it is shown in FIG. 6 in dotted lines, and in sliding in the medial direction, as it is shown in FIG. 7 in dotted lines.

The figures also show that the plateau 30 can receive against it lower face a shim or spacer 38 enabling compensation for possible bone ablations or absences at the support surfaces of the implant 3 against the tibia. This shim or spacer 38 comprises an opening 39 having a shape adapted to its engagement around the block 35 and the walls 37, as FIG. 2 shows it.

The intermediate plateau 4 is made of a slide promoting material, in particular made of a high density polyethylene. It comprises two glenoid side cavities 40 for reception of the lateral condyles 5, an anterior median eminence 41 designed to cooperate with the central condyle 9 of the femoral implant 2, a posterior bore 42 for reception of the socket 11 and the aforementioned groove 43 designed to cooperate with the anterior stud 32. The bore 42 enables a pivoting of the femoral implant 2-intermediate plateau 4 assembly about the axis of the stud 31, and the slot 43, centered on the axis of the bore 42, cooperates with the stud 32 to limit this pivoting to a sector delimited by the future positions of the stud 32 in abutment against the ends of this groove 43.

In practice, the medullar rods 21 are connected by screwing to the respective intermediate coupling parts 20, 34 without complete tightening then the suitable positions of the rods 21 are adjusted; the complete tightening of the rods 21 relative to the parts 20 and 34 is then carried out to immobilize these rods and these parts relative to the implants 2, 3.

As it appears from the foregoing, the invention provides a prosthesis having the decisive advantages of being fully adaptable to the specific anatomy of the joint to be treated, of thus having a reduced risk of wear and loosening, of being capable of being adaptable to a difficult arthroplasty, and having a high stability when the lower limb is extended.

It is evident that the invention is not limited to the form of implementation described above by way of example but it extends to all forms of implementation covered by the attached claims. It is thus that the term “medullar anchoring rod” must be understood in a broad sense, including short rods similar to “studs”, the plurality of angular positions of a medullar anchoring rod could be obtained by means of a ball joint type; the plurality of positions in translation could be obtained by sliding of a slide on a rail or in a slide.

1. A total knee prosthesis, comprising a femoral implant and a tibial implant equipped with medullar anchoring rods, and an intermediate plateau made of a material promoting sliding, in particular, comprising high density polyethylene, each medullar anchoring rod of an implant is not integral with the implant and is connectable to it, the prosthesis comprising to this end means for the coupling of each medullar anchoring rod to the corresponding implant and tightening means for immobilizing each medullar anchoring rod relative to the corresponding implant;
the prosthesis characterized in that the means that it comprises for the coupling of each medullar anchoring rod to the corresponding implant are designed to enable a plurality of possible angular positions of the medullar anchoring rod relative to the implant and a plurality of possible positions of this same medullar anchoring rod relative to the implant in translation along an axis contained in the frontal plane, that is to say lateral, or in the sagittal plane, that is to say, anteroposterior, of the implant.

2. A prosthesis according to claim 1, characterized in that the means for coupling of the femoral anchoring rod of the femoral implant are designed to enable angular adjustment of the anchoring rod in the frontal plane and the adjustment in translation of this rod in the sagittal plane.

3. A prosthesis according to claim 1, characterized in that the means for the coupling of the tibial anchoring rod to the tibial implant are designed to enable angular adjustment of the anchoring rod in the sagittal plane and the adjustment in translation of this rod in the frontal plane.

4. A prosthesis according to claim 1, characterized in that:
an anchoring rod is designed to be connected to the corresponding implant by an intermediate coupling part;
this intermediate coupling part has means for the coupling of the rod to it and a curved end wall;
the implant has a cavity for reception and retention of the aforementioned curved end wall, having the same profile as the aforementioned curved end wall and the bottom of which is delimited by a curved reception wall against which comes to bear the aforementioned curved end wall at the time of the coupling; this curved reception wall has an arc length greater than that of the aforementioned curved end wall, so that this curved end wall, and thus the aforementioned anchoring rod connected to it, can be positioned according to several angular positions relative to the implant; the cavity is shaped along an axis contained in the frontal plane or in the sagittal plane of the implant so that it enables the sliding of the aforementioned curved end wall in it in order to carry out the adjustment in translation of the medullar anchoring rod relative to the implant.

5. A prosthesis according to claim 4, characterized in that the means for the coupling of a medullar anchoring rod to an intermediary coupling part comprise:
a threaded bore longitudinally traversing the anchoring part and opening into the concave face of the aforementioned curved end wall;
a threaded rod integral with the medullar anchoring rod, adapted to be screwed into the aforementioned threaded bore, this rod having a length slightly greater than that of the bore so as, in coupling position of the medullar anchoring rod to the intermediate coupling part, to bear against the aforementioned curved reception wall and thus tighten the intermediate coupling part against the walls of the implant delimiting the aforementioned cavity for reception and retention of the aforementioned curved end wall.

6. A prosthesis according to claim 1, characterized in that it is a prosthesis having limited movement of the femoral implant relative to the tibial implant in translation in the sagittal plane, such as a prosthesis called “posterior-stabilized”, that is to say, comprising stops for limiting the aforementioned translation, or a prosthesis called “joint”, that is to say comprising means for mounting of the femoral implant relative to the tibial implant reducing the possibility of movement of the femoral implant relative to the tibial implant with a pivoting according to a determined transverse axis.

7. A prosthesis according to claim 1, characterized in that it comprises three condyles, that is to say that it has, at the femoral implant and at the intermediate plateau, curved median surfaces support arranged between the lateral condyles.

8. A prosthesis according to claim 1, characterized in that the intermediate plateau is movable relative to the tibial implant.

9. A prosthesis according to claim 8, characterized in that means are provided to limit the pivoting of the intermediate plateau relative to the tibial implant, in particular, a stud protruding from the plateau that forms the tibial implant and a groove in a circular arc centered on the pivot axis of the intermediate plateau, arranged in this intermediate plateau, this groove receiving the aforementioned stud and cooperating with it to limit the pivoting of the intermediate plateau relative to the tibial implant.

10. A prosthesis according to claim 1, characterized in that the femoral implant and/or tibial implant comprise means for mounting on them one or several shims or spacers enabling compensation for possible bone ablations or absences at the support surfaces of the implants against the femur and/or tibia.

11. Series of components enabling composition of the prosthesis according to claim 1, characterized in that it comprises:
a femoral implant, a tibial implant and an intermediate plateau;
two intermediate coupling parts;
two medullar anchoring rods assembled to the aforementioned intermediate coupling parts.

12. A series of components according to claim 11, characterized in that it comprises shims or spacers for compensation of bone ablations or absences, having different thicknesses.

13. A series of components according to claim 11 wherein the femoral and tibial components of different sizes include connecting means for the medullar anchoring rods of identical dimensions and shapes of one component to the other.

14. A series of components according to claim 11 wherein more than two medullar anchoring rods, include different diameters, lengths, shapes.
